(19) United States
(12) Patent Application Publication
(10) Pub. No.: US 2021/0299004 A1
(43) Pub. Date: Sep. 30, 2021

(54) MATTIFYING NAIL COMPOSITIONS

(71) Applicant: L'OREAL, Paris (FR)

(72) Inventors: Sam MORRISON, Clark, NJ (US); I-Fan HSIEH, Scotch Plains, NJ (US); Ramakrishnan HARIHARAN, Springfield, NJ (US)

(73) Assignee: L'OREAL, Paris (FR)

(21) Appl. No.: 16/835,984

(22) Filed: Mar. 31, 2020

(51) Int. Cl. A61K 8/02; A61K 8/25; A61K 8/19; A61K 8/96; A61Q 3/02

(52) U.S. Cl. CPC A61K 8/0279; A61K 8/25; A61Q 3/02; A61K 8/965; A61K 8/19

(57) ABSTRACT

Disclosed are anhydrous nail polish compositions that use hollow borosilicate particles as a mattifying agent. The hollow borosilicate particles may be, e.g., calcium aluminum borosilicate particles, and the particles may be present in concentrations of, e.g., between 0.1% and 5% by weight in the composition.

### FIELD OF THE INVENTION

The present invention relates to nail enamel compositions, and specifically mattifying nail enamel compositions comprising hollow borosilicate particles.

### BACKGROUND

Techniques for reducing gloss in skin care are commonplace. However, the expectations and requirements of gloss reduction for nail products are different than for skin care products. For example, literature for commercial mattifying materials often refers to “soft focus”, but this term applies for use on skin, not on nails.

Traditionally, matte properties have been provided to nail compositions by including silica or polylactic acid powders in the compositions. However, these techniques often result in less-than-satisfactory aesthetics. For example, when typical techniques are employed in a topcoat composition, the color of the underlying basecoat is tinted yellow. Thus, there remains a need for new ways to provide matte aesthetics in nail compositions.

### BRIEF SUMMARY

In a first aspect, the present invention is drawn to an anhydrous nail polish composition, the includes hollow borosilicate particles as a mattifying agent. Optionally, the hollow borosilicate particles comprise calcium. Optionally, the hollow borosilicate particles are calcium aluminum borosilicate particles. Optionally, the concentration of hollow borosilicate particles in the composition is between 0.1% and 5% by weight. Optionally, the hollow borosilicate particles have a mean particle diameter of between 9 μm and 13 μm. Optionally, the anhydrous nail polish composition is substantially free of silica, pearlescent borosilicate pigments, clay, or a combination thereof. Optionally, sufficient hollow borosilicate particles are present to reduce the gloss by an amount between 60% and 90%, as measured at 20°. Optionally, sufficient hollow borosilicate particles are present in an amount such that the gloss as measured at 60° is less than 7 times the gloss as measured at 20°. Optionally, the nail polish composition includes a primary film forming agent, a secondary film forming agent, a solvent, and/or a colorant. Optionally, the nail polish composition is substantially free of a colorant.

In a first aspect, the present invention is drawn to a method for coating a natural or synthetic nail. The method involves applying an anhydrous nail polish composition as described above over a natural or synthetic nail, then allowing the anhydrous nail polish composition to dry. Optionally, a second nail polish composition is first applied as a basecoat directly on the natural or synthetic nail, and the disclosed composition is used as a topcoat and is applied onto the basecoat. In such cases, the measured color of the natural or synthetic nail after application of the basecoat but prior to application of the anhydrous nail polish composition is substantially the same as a measured color of the natural or synthetic nail after the basecoat and anhydrous nail polish composition have been applied.

### DETAILED DESCRIPTION

As used herein, articles such as “a” and “an” when used in a claim, are understood to mean one or more of what is claimed or described.

As used herein, the term “about [a number]” is intended to include values rounded to the appropriate significant digit. Thus, “about 1” would be intended to include values between 0.5 and 1.5, whereas “about 1.0” would be intended to include values between 0.95 and 1.05.

As used herein, the term “anhydrous” means the compositions contain less than 1% water.

As used herein, the term “at least one” means one or more and thus includes individual components as well as mixtures/combinations.

As used herein, the terms “include”, “includes” and “including” are meant to be non-limiting.

As used herein, the term “substantially free [of a material]” means the material is present in an amount less than 100 ppm in the formulation.

As used herein, the term “substantially the same” color means that when a first color and a second color are viewed by an untrained observer, the observer perceives the same hue. Preferably, the two colors, when measured in the CIE L*a*b* color space, have a ΔE* value (where ΔE*=\sqrt{ΔL*+Δa*+Δb*}) of 6 or less.

The present invention is directed to an anhydrous nail polish composition. The composition comprises comprising a mattifying agent. The mattifying agent comprises a plurality of hollow borosilicate particles.

The hollow borosilicate particles according to the invention are typically homogeneous and essentially uniform in sphericity. While the mean particle diameter of the hollow borosilicate particles can range up to 100 μm, the mean particle diameter is typically between 3 μm and 20 μm, and preferably between 9 μm and 13 μm.

In some embodiments, the hollow borosilicate particles comprise an alkali metal or alkaline earth metal, such as calcium and/or sodium, and may optionally contain a transition metal or post-transition metal, such as aluminum. A preferred embodiment is calcium aluminum borosilicate particles. Other suitable the hollow borosilicate particles according to the present invention include those sodium borosilicate particulates marketed by PQ Corporation under the trademark Q-CEL. Also suitable are those calcium/sodium borosilicate hollow microspheres marketed by 3M.

Preferred hollow borosilicate particles are typically high slip, chemically inert, non-absorbent, non-porous free-flowing white powders. Preferred hollow borosilicate particles according to this invention include those marketed by Presperse Inc. under the trademark LUXSIL®.

In some embodiments, the hollow borosilicate particles have densities between 1.05 g/cc and 1.15 g/cc, have a weight loss on drying of less than 0.5%, and contain, e.g., less than 20 ppm of lead and less than 3 ppm of arsenic.

In some embodiments, the hollow borosilicate particles are present in the composition in an amount less than 10% by weight of the composition.

Preferred concentrations of the LUXSIL® free-flowing, spherical white powder microbeads are on the order of 0.1% to 10% by weight in the formulation. Concentrations from 0.1% to 5% by weight in the formulation are even more preferred.

In preferred embodiments, the anhydrous nail polish composition is substantially free of silica, pearlescent borosilicate pigments, clay, or a combination thereof.

The anhydrous nail polish composition will typically include other components, such as film formers, solvents, and/or colorants. In some embodiments, the composition is substantially free of any colorant.

Film Formers

Such other components may include one or more primary film forming agents (i.e., film forming agents present in an amount ≥50% by weight of all film formers in the composition), and may include one or more secondary film forming agents (i.e., film forming agents present in an amount <50% by weight of all film formers in the composition).

Film forming agents may be, e.g., cellulose-derived film formers, such as nitrocellulose, cellulose propionate, cellulose acetate butyrate, and hydroxyethylcellulose, hydroxypropylcellulose, methylcellulose, and ethylhydroxyethylcellulose. A suitable cellulose-derived film former may include, but is not limited to, those sold by EASTMAN™ Chemical, including but not limited to EASTMAN™ Cellulose Acetate Butyrate.

The film formers are typically present in a total amount ranging from about 1% to about 25% by weight.

Solvents

The compositions may comprise at least one oil or organic solvent. The compositions according to the invention may in particular comprise at least one oil chosen from at least one non-volatile oil, at least one volatile oil, and a mixture thereof.

Non-Volatile Oil

The term “oil” is intended to mean a fatty substance that is liquid at ambient temperature and at atmospheric pressure.

The term “non-volatile oil” is intended to mean an oil that remains on the nail at ambient temperature and pressure. More precisely, a non-volatile oil has an evaporation rate strictly less than 0.01 μmg/cm²/min. To measure this evaporation rate, 15 g of oil or of oil mixture to be tested are placed in a crystallizing dish 7 cm in diameter, which is placed on a balance in a large chamber of about 0.3 μm³ that is temperature-regulated, at a temperature of 25° C., and hygrometry-regulated, at a relative humidity of 50%. The liquid is allowed to evaporate freely, without stirring it, while providing ventilation by means of a fan (Papst-Motoren, reference 8550 N, rotating at 2700 rpm) placed in a vertical position above the crystallizing dish containing said oil or said mixture, the blades being directed towards the crystallizing dish, 20 cm away from the bottom of the crystallizing dish. The mass of oil remaining in the crystallizing dish is measured at regular intervals. The evaporation rates are expressed in mg of oil evaporated per unit of area (cm²) and per unit of time (minutes).

Said at least one non-volatile oil may be chosen from hydrocarbon-based oils and silicone oils, and mixtures thereof, preferably from hydrocarbon-based oils.

The non-volatile hydrocarbon-based oils that are suitable for the present invention may be chosen in particular from:

hydrocarbon-based oils of plant origin, such as triglycerides consisting of fatty acid esters of glycerol, the fatty acids of which may have chain lengths ranging from C4 to C28, these fatty acids possibly being linear or branched, and saturated or unsaturated; these oils are in particular wheatgerm oil, sunflower oil, grapeseed oil, sesame oil, corn oil, apricot oil, castor oil, shea oil, avocado oil, olive oil, soybean oil, sweet almond oil, rapeseed oil, cottonseed oil, hazelnut oil, macadamia oil, jojoba oil, palm oil, alfalfa oil, poppy oil, pumpkin oil, marrow oil, blackcurrant oil, evening primrose oil, millet oil, barley oil, quinoa oil, rye oil, safflower oil, candlenut oil, passion flower oil and musk rose oil; or alternatively caprylic/capric acid triglycerides such as those sold by the company Stéarineries Dubois or those sold under the names Miglyol 810@, 812@ and 818@ by the company Sasol;

synthetic ethers containing from 10 to 40 carbon atoms;

linear or branched hydrocarbons of mineral or synthetic origin other than the polymers according to the invention, such as petroleum jelly, polybutenes, polydecenes and squalane, and mixtures thereof;

synthetic esters such as the oils of formula R1COOR2 in which R1 represents the linear or branched fatty acid residue containing from 1 to 40 carbon atoms and R2 represents an in particular branched hydrocarbon-based chain containing from 1 to 40 carbon atoms, on condition that R1+R2 10, for instance purcellin oil (cetostearyl octanoate), isopropyl myristate, isopropyl palmitate, C12 to C15 alkyl benzoate, hexyl laurate, diisopropyl adipate, isononyl isononanoate, 2-ethylhexyl palmitate, isostearyl isostearate, alkyl or polyalkyl octanoates, decanoates or ricinoleates such as propylene glycol dioctanoate; hydroxylated esters such as isostearate lactate and diisostearyl malate; and pentaerythritol esters;

fatty alcohols that are liquid at ambient temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2-undecylpentadecanol; and

higher fatty acids such as oleic acid, linoleic acid or linolenic acid, and mixtures thereof.

Non-limiting examples of suitable non-volatile silicone oils that may be used in the composition in accordance with the invention may be non-volatile polydimethylsiloxanes (PDMSs), polydimethylsiloxanes comprising alkyl or alkoxy groups, that are pendent and/or at the end of a silicone chain, the groups each containing from 2 to 24 carbon atoms, phenyl silicones, such as phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates.

A composition optionally comprises at least one non-volatile hydrocarbon-based oil of plant origin, such as triglycerides consisting of fatty acid esters of glycerol the fatty acids of which may have chain lengths ranging from C4 to C28, in particular palm oil and hydrogenated jojoba oil. A composition is preferably free of silicone non-volatile oil(s).

A composition is preferably free of non-volatile oil. However, the total content of non-volatile oil(s) in a composition in accordance with the invention may range from 0.01% to 10% by weight, in particular from 0.1% to 8% by weight and preferably from 0.25% to 5% by weight relative to the total weight of the composition.

According to one preferred embodiment, a composition comprises less than 5% by weight of non-volatile oil(s) relative to the total weight of the composition.

Volatile Oil

The composition may comprise at least one volatile oil.

The term “volatile oil” is intended to mean an oil (or non-aqueous medium) that can evaporate upon application in less than one hour, at ambient temperature and atmospheric pressure. The volatile oil is a cosmetic volatile oil, which is liquid at ambient temperature. More specifically, a volatile oil has an evaporation rate of between 0.01 and 200 μmg/cm²/min, limits included.

This volatile oil may be hydrocarbon-based.

The volatile hydrocarbon-based oil may be chosen from hydrocarbon-based oils containing from 7 to 16 carbon atoms.

The composition may contain one or more volatile branched alkanes. The expression “one or more volatile branched alkanes” is intended to mean, without preference, “one or more volatile branched alkane oils”.

As a volatile hydrocarbon-based oil containing from 7 to 16 carbon atoms, mention may be made in particular of C8-C16 branched alkanes, such as C8-C16 isoalkanes (also known as isoparaffins), isododecane, isodecane, isohexadecane and for example the oils sold under the trade names Isopar or Permethyl, C8-C16 branched esters such as isohexyl neopentanoate, and mixtures thereof. Preferably, the volatile hydrocarbon-based oil containing from 8 to 16 carbon atoms is chosen from isododecane, isodecane and isohexadecane, and mixtures thereof, and is in particular isododecane.

The composition may contain one or more volatile linear alkanes. The term “one or more volatile linear alkanes” is intended to mean, without preference, “one or more volatile linear alkane oils”.

A volatile linear alkane that is suitable for the invention is liquid at ambient temperature (about 25° C.) and at atmospheric pressure (760 μmmHg).

A “volatile linear alkane” that is suitable for the invention is intended to mean a cosmetic linear alkane, which is capable of evaporating upon application in less than one hour, at ambient temperature (25° C.) and atmospheric pressure (760 mmHg), which is liquid at ambient temperature, in particular having an evaporation rate ranging from 0.01 to 15 μmg/cm²/min, at ambient temperature (25° C.) and atmospheric pressure (760 μmmHg).

The linear alkanes, preferably of plant origin, comprise from 7 to 15 carbon atoms, in particular from 9 to 14 carbon atoms and more particularly from 11 to 13 carbon atoms.

Non limiting examples of suitable alkanes are described in patent applications WO 2007/068,371 or WO 2008/155,059 by the company Cognis (mixtures of distinct alkanes that differ by at least one carbon). These alkanes are obtained from fatty alcohols, which are themselves obtained from coconut oil or palm oil.

As examples of linear alkanes that are suitable for the invention, mention may be made of n-heptane (C7), n-octane (C8), n-nonane (C9), n-decane (C10), n-undecane (C11), n-dodecane (C12), n-tridecane (C13), n-tetradecane (C14) and n-pentadecane (C15), and mixtures thereof, and in particular the mixture of n-undecane (C11) and n-tridecane (C13) described in Example 1 of patent application WO 2008/155 059 by the company Cognis. Mention may also be made of n-dodecane (C12) and n-tetradecane (C14) sold by Sasol under the references, respectively, Parafol 12-97 and Parafol 14-97, and also mixtures thereof.

The linear alkane may be used alone or as a mixture of at least two distinct alkanes that differ from each other by a carbon number of at least 1, and in particular a mixture of at least two linear alkanes comprising from 10 to 14 distinct carbon atoms that differ from each other by a carbon number of at least 2, and in particular a mixture of C11/C13 volatile linear alkanes or a mixture of C12/C14 linear alkanes, in particular an n-undecane/n-tridecane mixture.

As a variant or additionally, the composition prepared may comprise at least one volatile silicone oil or solvent that is compatible with cosmetic use.

The term “silicone oil” is intended to mean an oil containing at least one silicon atom, and in particular containing Si—O groups. According to one embodiment, said composition comprises less than 10% by weight of non-volatile silicone oil(s), relative to the total weight of the composition, better still less than 5% by weight, or even is free of silicone oil.

Volatile silicone oils that may be mentioned include cyclic polysiloxanes and linear polysiloxanes, and mixtures thereof. Volatile linear polysiloxanes that may be mentioned include hexamethyldisiloxane, octamethyltrisiloxane, decamethyltetrasiloxane, tetradecamethylhexasiloxane and hexadecamethylheptasiloxane. Volatile cyclic polysiloxanes that may be mentioned include hexamethylcyclotrisiloxane, octamethylcyclotetrasiloxane, decamethylcyclopentasiloxane and dodecamethylcyclohexasiloxane.

A composition is preferably free of non-volatile oil.

In some embodiments, at least one volatile oil, and preferably a volatile hydrocarbon-based oil, may be present in a total amount of at least about 30% by weight, such as between about 30% to about 60% by weight.

According to one embodiment, a composition comprises less than 5% by weight of volatile oil(s) relative to the total weight of the composition.

Colorants

The disclosed compositions may include one or more colorants.

Colorants are preferably chosen from pulverulent materials, liposoluble dyes and water-soluble dyes, and mixtures thereof.

Preferably, the compositions according to the invention comprise at least one pulverulent colorant. The pulverulent colorants may be chosen from pigments and nacres, and preferably from pigments.

The pigments may be white or colored, inorganic and/or organic, and coated or uncoated. Among the inorganic pigments, mention may be made of metal oxides, in particular titanium dioxide, optionally surface-treated, zirconium, zinc or cerium oxide, and also iron, titanium or chromium oxide, manganese violet, ultramarine blue, chromium hydrate and ferric blue. Among the organic pigments that may be mentioned are carbon black, pigments of D&C type and lakes based on cochineal carmine or on barium, strontium, calcium or aluminum.

The nacres may be chosen from white nacreous pigments such as mica coated with titanium or with bismuth oxychloride, colored nacreous pigments such as titanium mica with iron oxides, titanium mica with in particular ferric blue or chromium oxide, titanium mica with an organic pigment of the abovementioned type, and also nacreous pigments based on bismuth oxychloride.

The liposoluble dyes are, for example, Sudan Red, D&C Red 17, D&C Green 6, ß-carotene, soybean oil, Sudan Brown, D&C Yellow 11, D&C Violet 2, D&C Orange 5, quinoline yellow and annatto.

Preferably, the pigments contained in the compositions according to the invention are chosen from metal oxides.

These colorants may be present in a content ranging from 0.01% to 30% by weight relative to the total weight of the composition and in particular from 6% to 22% by weight relative to the total weight of the composition.

Preferably, the colorant(s) is (are) chosen from one or more metal oxides that are present in a content of greater than or equal to 2% by weight relative to the total weight of the composition, and advantageously inclusively between 6% and 22% by weight relative to the total weight of the composition.

The above-described anhydrous nail polish composition may be used to coating natural or synthetic nails. The anhydrous nail polish composition is applied over a natural or synthetic nail, then allowed to dry.

In some embodiments, a second nail polish composition is used as a basecoat and is directly applied to the natural or synthetic nail, and then the presently disclosed anhydrous nail polish composition is applied directly onto the basecoat. In such embodiments, the presently disclosed anhydrous nail polish composition is preferably configured to an amount of the hollow borosilicate particles such that the underlying basecoat color is substantially the same before and after the application of the presently disclosed anhydrous nail polish composition.

In some embodiments, the anhydrous nail polish composition is directly applied to the natural or synthetic nail. In such cases, the addition of the hollow borosilicate particles should result in the formulation having substantially the same color as before the particles were added.

In some embodiments, the hollow borosilicate particles are present in an amount sufficient to reduce gloss as measured at 20° by an amount between 60% and 99%, such as between 60% and 98%, between 60% and 90%, or between 80% and 90%. In other embodiments, the hollow borosilicate particles are present in an amount such that the gloss as measured at 60° is less than 7 times the gloss as measured at 20°.

### Example 1

A standard nail polish formulation base was used for comparing two formulations: an inventive formulation where 4% by weight of hollow calcium aluminum borosilicate particles were added to the base, and a comparative formula where 4% by weight of kaolin was added to the base.

The standard nail polish formulation base comprised 6% nitrocellulose, 8% plasticizers, and the remainder being ethyl acetate.

Batches of each composition were manufactured according to standard procedures. Films were then drawn down onto Laneta Form 5C—Opacity cards and allowed to dry. Gloss measurements were made using a BYK micro-TRI-gloss meter. The results, shown in Table 1, below, are at the 20° and 60° reflectance angles. As can be seen, there is significantly less change in gloss (between the 20° and 60® angles) using calcium aluminum borosilicate particles as opposed to using kaolin.

TABLE 1
Example 1 Gloss Results.

| INCI Name | Gloss at 20° | Gloss at 60° |
|---|---|---|
| Kaolin | 1.6 | 13 |
| Hollow Calcium Aluminum | 1.3 | 8.8 |
| Borosilicate Particles | | |

### Example 2

The above-described standard nail polish formulation base was used for comparing various formulations. The inventive formulation where up to 2% by weight of hollow calcium aluminum borosilicate particles were added to the base, a comparative formula where 2% by weight of silica was added to the base, and comparative formulations where pearl pigments (plates of borosilciate with titanium dioxide) are added to the base.

Batches of each composition were manufactured according to standard procedures. Films were then drawn down onto Laneta Form 5C—Opacity cards and allowed to dry. Gloss measurements were made using a BYK micro-TRI-gloss meter. The results, shown in Table 2, below, are at the 20° reflectance angles.

TABLE 2
Evaluation Results for 8 Formulas.

| # | Formula | Gloss at 20° |
|---|---|---|
| 1 | Base | 59 |
| 2 | Base + 0.75% by weight hollow Ca Al borosilicate | 23 |
| 3 | Base + 1.50% by weight hollow Ca Al borosilicate | 9.7 |
| 4 | Base + 2.00% by weight hollow Ca Al borosilicate | 6.2 |
| 5 | Base + 2.00% by weight Silica | 0.5 |
| 6 | Base + 0.75% Pearl Pigment | 53 |
| 7 | Base + 1.50% Pearl Pigment | 46 |
| 8 | Base + 2.00% Pearl Pigment | 34 |

Surprisingly, the hollow calcium aluminum borosilicate particles performed significantly better at mattifying the formulation than the plates of calcium aluminum borosilicate and titanium dioxide. Further, even at low usages, the hollow calcium aluminum borosilicate particles provided reductions in gloss as measured at 200-0.75% by weight had over a 60% reduction in gloss, and a 2.00% by weight had just under a 90% reduction in gloss.

### Example 3

The standard nail polish formulation base described above was used for comparing two formulations: an inventive formulation where 4% by weight of hollow calcium aluminum borosilicate particles were added to the base, and a comparative formula where 4% by weight of silica was added to the base.

Batches of each composition were manufactured using standard procedures. Samples were poured into standard nail polish bottles fitted with brush and cap. LabCh measurements were obtained with Verivide's DigiEye system. The results are shown below, in Table 3.

TABLE 3
LabCh Color Measurements.

| # | Formula | L* | a* | b* | C* | h |
|---|---|---|---|---|---|---|
| 1 | Base + 4.00% by weight | 83.94 | 1.03 | 11.4 | 11.44 | 84.84 |
| | Silica | | | | | |
| 2 | Base + 4.00% by weight | 90.33 | 0.02 | 8.32 | 8.32 | 89.84 |
| | hollow Ca Al borosilicate | | | | | |

As can be seen in Table 3, the use of silica results in a darker (lower L* value), very slightly redder (higher a* value), and more yellowish color (higher b* value) than with the hollow calcium aluminum borosilicate particles. These two colors are not substantially the same. In addition to being a visually noticeable coloration difference, the ΔE* value between these color measurements is about 7.17.

Those skilled in the art will recognize or be able to ascertain using no more than routine experimentation many equivalents to the specific embodiments of the invention described herein. Such equivalents are intended to be encompassed by the following claims.

What is claimed is:

1. An anhydrous nail polish composition comprising:
a mattifying agent, wherein the mattifying agent comprises a plurality of hollow borosilicate particles.

2. The anhydrous nail polish composition according to claim 1, wherein the plurality of hollow borosilicate particles comprise calcium.

3. The anhydrous nail polish composition according to claim 1, wherein the plurality of hollow borosilicate particles are calcium aluminum borosilicate particles.

4. The anhydrous nail polish composition according to claim 1, wherein the plurality of hollow borosilicate particles are present in the anhydrous nail polish composition in an amount between 0.1% and 5% by weight of the anhydrous nail polish composition.

5. The anhydrous nail polish composition according to claim 1, wherein the plurality of hollow borosilicate particles has a mean particle diameter of between 9 μm and 13 μm.

6. The anhydrous nail polish composition according to claim 1, wherein the anhydrous nail polish composition is substantially free of silica, pearlescent borosilicate pigments, clay, or a combination thereof.

7. The anhydrous nail polish composition according to claim 1, wherein the plurality of hollow borosilicate particles are present in an amount sufficient to reduce gloss as measured at 20° by an amount between 60% and 90%.

8. The anhydrous nail polish composition according to claim 1, wherein the plurality of hollow borosilicate particles are present in an amount such that the gloss as measured at 60° is less than 7 times the gloss as measured at 20°.

9. The anhydrous nail polish composition according to claim 1, further comprising at least one primary film forming agent.

10. The anhydrous nail polish composition according to claim 1, further comprising at least one secondary film forming agent.

11. The anhydrous nail polish composition according to claim 1, further comprising at least one solvent.

12. The anhydrous nail polish composition according to claim 1, further comprising at least one colorant.

13. The anhydrous nail polish composition according to claim 1, wherein the composition is substantially free of a colorant.

14. A method for coating a natural or synthetic nail, comprising the steps of:
applying an anhydrous nail polish composition according to claim 1 over a natural or synthetic nail; and
allowing the anhydrous nail polish composition to dry.

15. The method according to claim 15, further comprising applying an additional nail polish composition as a basecoat directly on the natural or synthetic nail, wherein a measured color of the natural or synthetic nail after application of the basecoat but prior to application of the anhydrous nail polish composition is substantially the same as a measured color of the natural or synthetic nail after the basecoat and anhydrous nail polish composition have been applied.

16. The method according to claim 13, wherein the plurality of hollow borosilicate particles comprise calcium.

17. The method according to claim 13, wherein the plurality of hollow borosilicate particles are calcium aluminum borosilicate particles.

18. The method according to claim 13, wherein the plurality of hollow borosilicate particles are present in the anhydrous nail polish composition in an amount between 0.1% and 5% by weight of the anhydrous nail polish composition.

19. The method according to claim 13, wherein the anhydrous nail polish composition is substantially free of silica, pearlescent borosilicate pigments, clay, or a combination thereof.
